(19) United States
(12) Patent Application Publication
(10) Pub. No.: US 2017/0143743 A1
(43) Pub. Date: May 25, 2017

(54) FORMULATION FOR ORAL ADMINISTRATION CONTAINING MESALAZINE

(71) Applicant: VALPHARMA INTERNATIONAL S.P.A., Pennabilli (IT)

(72) Inventors: Roberto VALDUCCI, Savignano Sul Rubicone (IT); Serozh AVANESSIAN, Villa Verucchio (IT)

(21) Appl. No.: 15/319,634

(22) PCT Filed: Jun. 15, 2015
(86) PCT No.: PCT/IB2015/054506

§ 371 Date: Dec. 16, 2016

(30) Foreign Application Priority Data
Jun. 16, 2014 (IT) FI2014A000148

(51) Int. Cl. A61K 31/606; A61K 9/50; A61K 9/00; A61K 9/16

(52) U.S. Cl. CPC A61K 31/606; A61K 9/1635; A61K 9/1623; A61K 9/5073; A61K 9/5026; A61K 9/5047; A61K 9/5015; A61K 9/5089; A61K 9/1688; A61K 9/0053

(57) ABSTRACT

The present invention relates to a formulation containing Mesalazine in a stable form, obtained by coating the starting core with a first membrane in non-aqueous solution; said core containing Mesalazine at a high concentration, greater than 90%, obtained by producing starting cores with Active Substance content greater than 97%. The formulation according to the present invention containing Mesalazine with specific release in the colon; in particular the capsule formulation containing 500 mg of Mesalazine with specific release in the colon.

### FIELD OF THE INVENTION

The present invention relates to the pharmaceutical compositions field, in particular it relates to formulations for the oral administration of Mesalazine in multi-particulate, multi-layer form.

### BACKGROUND OF THE INVENTION

Mesalazine, also known as 5-Aminosalicylic Acid, generally abbreviated as 5-ASA, is an Active Substance that is now widely known in that it now is the best medicinal product in the treatment of inflammatory states of the colon, and is the best adjuvant in maintenance and improvement therapies of ulcerative colitis and Crohn's disease. As Acetylsalicylic Acid derivative, it maintains its anti-inflammatory characteristics but has the advantage of acting only at topical intestinal level, avoiding systemic absorption; hence the need to formulate preparations that have a specific release only at the area of action, i.e. the colon.

Present on the market in different pharmaceutical forms, it is characterised by having high doses (but low concentration of the Active Substance, generally equal to 500-550 mg of Mesalazine per g of formulation), and formulations containing 1-2 g of the Active Substance are frequent. This very often worsens patient compliance in taking the medicinal product, as the total quantity of medicinal product is either too high, or divided into several doses. The object of the present invention is thus to provide a formulation having a high concentration of Mesalazine, to decrease the total quantity to be taken and to improve patient acceptability.

EP1178781B1 describes a formulation containing Mesalazine that is exclusively released in the colon. It is a multi-layer formulation comprising:

a core containing the Active Substance, an inner membrane containing a pH-independent retardant polymer and an outer membrane containing a pH-dependent polymer that exclusively releases in the colon.
Core: contains about 50% API; and consists of neutral granules (0.5-0.6 mm) that are coated, by spraying in a Fluid Bed apparatus, with a layer containing API (Mesalazine, Lactose and Aerosil) and a binder (Kallidon 25 and water).
Inner Membrane: membrane comprising a polymer or a mixture of methacrylic acid-derived polymers (Eudragit RS, Eudragit RL), with the characteristic of having a dissolution that is slow and independent of the pH.
Outer Membrane: membrane comprising a polymer or a mixture of methacrylic acid-derived polymers (Eudragit FS 30 D), with the characteristic of having a dissolution that is pH-dependent, exclusively in of the colon.

Another problem often found in formulations containing Mesalazine, is ensuring stability of the Active Substance over time; the development of impurities during stability studies is indeed frequent. The impurities are generally due to the oxidation that occurs when the Active Substance remains in contact with water. In fact, the membranes used to confer topical release, are suspended in water, a small part of which remains present in the formulation even after drying. The cause of the degradation of Mesalazine over time is this very water. The object of the present invention is to provide a formulation that can overcome this problem, guaranteeing the stability of Mesalazine over time while ensuring topical intestinal absorption in the colon.

### SUMMARY OF THE INVENTION

The present invention resolves the above-mentioned-mentioned problems by means of granules containing Mesalazine as Active Pharmaceutical Ingredient (API), said granules consisting of API in mixture with a dried gelled composition in a ratio of between 97:3 and 99:1, referring to the dry portion of the composition; said granules being obtained by extrusion, spheronization and drying of a mixture of API with a gelled composition consisting of a mixture of 5-10% Polyvinylpyrrolidone, 20-40% Polysorbate and 45-75% Water, where the % relate to the percentages by weight with respect to the total weight of the gelled composition.

The granules in question are produced according to the extrusion+spheronization technique; the procedures known to the state of the art include the use of a percentage of plastic matrix, such as microcrystalline Cellulose, ranging between 30% and 50%; this gives the compound a suitable consistency for being extruded and spheronized. From here it can be deduced that the titre of the granules will be low and may not exceed 70%.

It has however been surprisingly found that by mixing just the API with the gel, in a ratio ranging between 97:3 and 99:1, preferably between 97.5:2.5 and 98.5:1.5, even more preferably 98:2 (relating to the dry weight of the substance), to then extrude and spheronize the compound obtained, compact granules with a high titre are obtained without the need to add plastic material.

Another surprising finding is that the gelled composition gives the compound a plasticity and malleability such as to be extruded with a very small mesh, so as to be able to obtain granules with a very low granulometry that are generally not obtainable according to traditional state of the art techniques. The density of the granules, which reaches 0.89 g/ml, is thus also increased with the consequent possibility of inserting in a capsule a large amount of Mesalazine per unit volume. Moreover, the gelled composition in the granules according to the invention increases the binding effect and improves the stability of the API, acting as antioxidant over time; in fact, contrary to other formulations that require the addition of anti-oxidants, a formulation comprising the granules according to the invention maintains stability over time without the addition of further ingredients. It is therefore understood that the use of polysorbate in the pellet formulation is not only merely one of the many possible excipients known in the state of the art, but has a fundamental importance, as it serves to create the gelling solution, which not only allows pellets having a very high titre to be produced, but is also responsible for the stability thereof over time.

It is also surprising that the water used for the gelled composition does not disturb the stability of the API. After extrusion and spheronization, the granules are dried immediately and in a prolonged manner to eliminate all of the water, nonetheless a very small fraction nevertheless remains within them. However, it has been surprisingly noted that the granules maintain an unexpected stability over time, produced by the antioxidant effect of the gelling substance used for granulation. Thus in one aspect, the present invention relates to a pharmaceutical formulation comprising the above-mentioned granules, it in particular relates to multi-layer pellets comprising the afore-mentioned granules as inner core.

Multi-layer pellets according to the invention comprise:
an inner core consisting of the above-mentioned granules;
a first inner coating membrane that surrounds and comes into contact with the core, said first membrane being pH-independent comprising a cellulose-derived polymer, dissolved in a non-aqueous solvent;
a second outer coating membrane that surrounds the first membrane, said second membrane being gastroprotective pH-dependent comprising a methacrylic acid derivative.

To overcome the problem of the development of impurities caused by the residual water deriving from the gastro-resistant coats, the multi-layer pellets of the present invention are characterised by a first protective coat of the cores with a membrane in a non-aqueous solution. The fact that the first coating is applied in a non-aqueous environment combined with the surprising stability of the cores produced by the gelling substance, leads to an even higher final stability, since there is no contact with water during coating.

The above-mentioned pellets are externally coated with a methacrylic acid derivative (generally Eudragit FS 30 D) to guarantee a release of the API exclusively in the colon; in fact, the chemical structure of the polymer ensures that it only dissolves in environments with a pH greater than 7.2, and this is only found in the last part of the intestine. The multi-layer pellets will pass through both the stomach and the first part of the intestine intact, to then release the medicinal product only in the last section.

An unexpected synergistic interaction between the two membranes has been surprisingly noted. Indeed, in addition to the protective effect of the first membrane and the gastro-resistant effect of the second membrane, it has been found that the two act together to regulate the release profile.

Ethyl cellulose being a pH-independent retardant polymer, it also acts here as a retardant in the release of the Active Substance, trapping for a certain period of time most of the Mesalazine within the pellets, for a period which fully or partially coincides with the time it takes the intestine to carry the medicinal product to the colon. The topical release effect in the colon, produced by the Eudragit of the second membrane, is also added to this retarding effect. An exclusive release of Mesalazine in the site of action is thus obtained. In addition thereto, the greatest advantage is that, the medicinal product having already been retarded by the first membrane, the amount of second membrane to be applied will be extremely reduced, thus leading to a greater final titre of the pellets being obtained. This aspect, together with the preceding concept of starting cores having an extremely high titre, leads to a finished product with a surprisingly high amount of Mesalazine, higher than 90%, being obtained.

Thus in one aspect, the present invention relates to a pharmaceutical formulation comprising the above-mentioned multi-layer pellets; in particular a pharmaceutical formulation comprising the above-mentioned pellets.

### DETAILED DESCRIPTION OF THE INVENTION

For one aspect, the present invention relates to a process for preparing the granules according to the invention, said process comprising preparing the gelled composition by first dissolving Polyvinylpyrrolidone in water and then, on dissolution, adding Polysorbate to achieve the gelification. The gelled composition is indeed a dense composition with viscosity ranging between 4800 mP*s and 5200 mP*s.

The process for preparing the granules also comprises that the above-mentioned gelled composition be added to and mixed with the Mesalazine, preferably in a Z-arm kneader; the compound is at this point extruded, spheronized and dried. Drying is preferably carried out in a Fluid Bed by means of the inlet of hot air at 80° C. until the product reaches 50° C. The result is a starting core containing Mesalazine in a percentage greater than 97%.

Extrusion preferably takes place with a net having mesh 400-600 μm mesh.

The granules obtained according to the invention have an average size ranging between 0.4 mm and 2 mm in diameter, preferably 0.45 mm-1.5 mm, more preferably 0.5 mm-0.8 mm.

The first protective membrane comprises a polymer, preferably Ethyl cellulose having a viscosity of between 3 and 110 cps, and a solvent, preferably Acetone, Ethanol and mixtures thereof; plasticisers, such as Triethyl citrate, Dibutyl sebacate or Polyethylene glycol and anti-sticking agents, such as Talc, Magnesium stearate, colloidal anhydrous silica or sodium stearyl fumarate, can be optionally present.

The viscosity of the Ethyl cellulose was calculated on 5% solutions in Toluene/Ethanol (80%:20%) measured at 25° C. in a Ubbelohde viscometer.

The polymer is present in solution in a percentage ranging between 1% and 10%, preferably between 3% and 8%, more preferably still between 5% and 6%.

The pellets are coated with the first membrane in a Fluidized Bed or in a Coating Pan until an increase in weight ranging between 0.2% and 2% of the dry weight is achieved with respect to the weight of the cores, preferably 0.5%-1.5%, even more preferably 0.8%-1.2%.

The second outer, gastroprotective membrane therefore comprises a methacrylic acid derivative, and water; plasticisers, such as Triethyl citrate and Polysorbate, and anti-sticking agents, such as talc and glyceryl monostearate, can be optionally present. The methacrylic acid derivative is selected from anionic polymers with methacrylic acid as functional group such as, for example, Eudragit L100-55, Eudragit L 30 D-55, Eudragit L100, Eudragit L12.5, Eudragit S100, Eudragit S12.5, Eudragit FS 30 or mixtures thereof; they can sometimes also be mixed with Eudragit NE30D and Eudragit NE40D to adjust the gastro-resistance thereof.

The methacrylic acid derivative is present in suspension in a percentage ranging between 12% and 28%, preferably between 15% and 25%, preferably between 16% and 20%.

The pellets are coated with the second membrane in a Fluidized Bed or in a Coating Pan until an increase in weight ranging between 5% and 15% of the dry weight is achieved with respect to the weight of the cores, preferably 6%-12%, even more preferably 7%-9%.

The pellets can at this point be sold in bags or, given the high percentage of Active Substance, even encapsulated or compressed.

The present invention therefore relates to a pharmaceutical formulation comprising the above-mentioned multi-layer pellets, said formulation in the form of tablets or capsules.

The pellets of the invention can safely be mixed with Cellulose, and compressed with 400 mg-500 mg-800 mg dosages.

Particularly advantageous, however, is the possibility of encapsulating in a single dose an amount of Mesalazine equal to 500 mg, which is the generally most common daily dose. Unlike the other formulations present in literature, these pellets, having a medium to high titre (>90%) and a high density (>0.89 g/ml or 900 mg/g), have a very low specific weight per unit of Active Substance and can therefore can be inserted into a 500 mg size 0 Capsule without any problems. This avoids the problem of multi-dose administrations, which would worsen patient compliance.

A preferred embodiment of the invention provides for the encapsulation of 500 mg of pellets in a single capsule.

The present invention will be better understood in the light of the following embodiments.

### EXPERIMENTS

### Example A—Classic Reference Formulation of the State of the Art

The composition of SALOFALK® GRANU-STIX, also indicated in the patent under the name of CLASSIC FORMULATION, is specified hereunder.

SALOFALK RTM GRANU-STIX 3 g

| COMPONENT | % |
|---|---|
| Mesalazine | 53.85% |
| Aspartame | 43.15% |
| Croscarmellose sodium | |
| Citric acid | |
| Colloidal silica | |
| HPMC | |
| Magnesium stearate | |
| Eudragit L100 | |
| Methylcellulose | |
| Microcrystalline cellulose | |
| Eudragit NE40D (contains 2% Nonoxynol | |
| 100) | |
| Polyvinylpyrrolidone | |
| Simethicone | |
| Ascorbic acid | |
| Talc | |
| Titanium dioxide | |
| Triethyl citrate | |
| Vanilla flavour (contains PEG) | |

### Example 1

Core

The following gelled composition is prepared:

| COMPONENT | AMOUNT (g) |
|---|---|
| Polyvinylpyrrolidone | 100 |
| Polysorbate | 320 |
| Water | 580 |
| Total | 1000 |

dissolving Polyvinylpyrrolidone in water, to then add Polysorbate on dissolution. Mesalazine raw material is mixed with the above-mentioned gelled composition to then extrude (500 μm net) and spheronize the compound. Immediately following spheronization, the granules obtained are dried in a Fluid Bed with inlet air at 80° C., up to a product temperature of 50° C. Cores having an average diameter of 480 μm-520 μm are obtained with the following dry matter composition:

| COMPONENT | AMOUNT (g) |
|---|---|
| Mesalazine | 980 |
| Polyvinylpyrrolidone | 4.76 |
| Polysorbate | 15.24 |
| Total | 1000 |

First Protective Coating

A protective membrane is prepared, comprising as follows:

| COMPONENT | AMOUNT (g) |
|---|---|
| Ethyl cellulose | 50 |
| Triethyl citrate | 1 |
| Acetone | 949 |
| Total | 1000 |

Dissolving Ethyl cellulose in Acetone, then adding Talc on dissolution.

The protective membrane is sprayed onto the previously obtained cores using a Glatt Fluid Bed with Wurster insert, up to a weight increase of 1% with respect to the initial weight of the pellets, thus obtaining a compound with a Mesalazine content of 97.03%.

Second Gastro-Resistant Coating

A protective membrane is prepared, comprising as follows:

| COMPONENT | AMOUNT (g) |
|---|---|
| Eudragit FS 30 D | 180 |
| Polysorbate | 3 |
| Triethyl citrate | 9 |
| Glyceryl monostearate | 7 |
| Water | 801 |
| Total | 1000 |

Homogenising Polysorbate, Triethyl citrate and Glyceryl monostearate in water at 80° C., then adding Eudragit once the compound has cooled.

The gastro resistant membrane is sprayed onto the previously obtained cores using a Glatt Fluid Bed with Wurster insert, up to a weight increase of 7% with respect to the initial weight of the pellets, thus obtaining a compound with a Mesalazine content of 90.68%.

The Finished Product has the following characteristics:

| | | |
|---|---|---|
| Titre | 90.68% | |
| Average diameter | d(50) = 470 μm | d(90) = 490 μm |
| Density | 0.84 g/ml | |

The multilayer pellets thus obtained are analysed in-vitro in HCl 0.1N/750 ml for 2 h, and then adjusted to pH 7.2/1000 ml; the following dissolution profile is obtained:

| | PERCENTAGE | | |
|---|---|---|---|
| | RELEASE of the | PERCENTAGE | DIS- |
| | multi-layer pellets | RELEASE of | SOLUTION |
| | according to the | SALOFALK ® | SPECIFI- |
| TIMEFRAMES | invention | GRANU-STIX ® | CATIONS |
| 2 h in HCl 0.1N | 0% | 0.5% | <10% |
| pH ADJUSTMENT | | | |
| 30′ in pH 7.2 | 68.1% | 61.4% | N.L.T. 60% |
| 60′ in pH 7.2 | 98.0% | 86.9% | N.L.T. 85% |

Stability studies of the pellets according to the present invention are set up, in a climatic chamber with Temperature of 25° C. and Relative Humidity of 60%, to verify the development of impurities. After 3 to 6 months the following results are obtained and are compared with the stability results of a formulation obtained according to traditional composition and production methods:
analysis at 3 months (25° C.-60% RH):

| | | FORMULATION | CLASSIC SALOFALK ® |
|---|---|---|---|
| | | according to | GRANU-STIX ® |
| IMPURITIES | LIMITS | the invention | FORMULATION |
| Known | 0.5% | 0.2% | 0.4% |
| Unknown | 1% | 0.3% | 1.3% |
| Totals | 1.5% | 0.5% | 1.7% |

analysis at 6 months (25° C.-60% RH):

| | | FORMULATION | CLASSIC |
|---|---|---|---|
| IMPURITIES | LIMITS | according to the invention | FORMULATION |
| Known | 0.5% | 0.4% | 0.6% |
| Unknown | 1% | 0.6% | 1.1% |
| Totals | 1.5% | 1.0% | 1.8% |

### Example 2

Core

The following gelled composition is prepared:

| COMPONENT | AMOUNT (g) |
|---|---|
| Polyvinylpyrrolidone | 80 |
| Polysorbate | 300 |
| Water | 620 |
| Total | 1000 |

dissolving Polyvinylpyrrolidone in water, to then add Polysorbate on dissolution. Mesalazine raw material is mixed with the above-mentioned-mentioned solution, to then extrude (400 μm net) and spheronize the compound. Immediately following spheronization, the granules obtained are dried in a fluid bed with inlet air at 80° C., up to a product temperature of 50° C. Cores having an average diameter of 380 μm-430 μm are obtained with the following dry matter composition:

| COMPONENT | AMOUNT (g) |
|---|---|
| Mesalazine | 990 |
| Polyvinylpyrrolidone | 2.10 |
| Polysorbate | 7.90 |
| Total | 1000 |

First Protective Coating

A protective membrane is prepared, comprising as follows:

| COMPONENT | AMOUNT (g) |
|---|---|
| Ethyl cellulose | 50 |
| Talc | 2.5 |
| Acetone | 947.5 |
| Total | 1000 |

Dissolving Ethyl cellulose in Acetone, then adding Talc on dissolution.

The protective membrane is sprayed onto the previously obtained cores using a GS Automatic Coating Pan, up to a 1.2% increase in weight with respect to the initial weight of the pellets, thus obtaining a compound with a 97.83% Mesalazine content.

Second Gastro-Resistant Coating

A protective membrane is prepared, comprising as follows:

| COMPONENT | AMOUNT (g) |
|---|---|
| Eudragit FS 30 D | 160 |
| Polysorbate | 3 |
| Triethyl citrate | 9 |
| Glyceryl monostearate | 7 |
| Water | 821 |
| Tot. | 1000 |

Homogenizing Polysorbate, Triethyl citrate and Glyceryl monostearate in water at 80° C., then adding Eudragit once the compound has cooled.

The gastro-resistant membrane is sprayed onto the previously obtained cores using a GS Automatic Coating Pan, up to a 7.5% increase in weight with respect to the initial weight of the pellets, thus obtaining a compound with a 91.00% Mesalazine content.

The Finished Product has the following characteristics:

| | |
|---|---|
| Titre | 91.00% |

| | | |
|---|---|---|
| Average diameter | d(50) = 386 μm | d(90) = 429 μm |

| | |
|---|---|
| Density | 0.89 g/ml |

The pellets are analysed in-vitro in HCl 0.1N/750 ml for 2 h, and then adjusted to pH 7.2/1000 ml; the following dissolution profile is obtained:

| | PERCENTAGE | |
|---|---|---|
| | RELEASE of the | |
| | multi-layer pellets | |
| | according to | DISSOLUTION |
| TIMEFRAMES | the invention | SPECIFICATIONS |
| 2 h in HCl 0.1N | 0.3% | <10% |
| pH ADJUSTMENT | | |
| 30′ in pH 7.2 | 79.4% | N.L.T. 60% |
| 60′ in pH 7.2 | 99.2% | N.L.T. 85% |

Stability studies of the pellets according to the present invention are set up, in a climatic chamber with Temperature of 25° C. and Relative Humidity of 60%, to verify the development of impurities. After 3 to 6 months the following results are obtained and are compared with the stability results of a formulation obtained according to traditional composition and production methods:
analysis at 3 months (25° C.-60% RH):

| | | FORMULATION | |
|---|---|---|---|
| | | according to | CLASSIC |
| IMPURITIES | LIMITS | the invention | FORMULATION |
| Known | 0.5% | 0.3% | 0.4% |
| Unknown | 1% | 0.2% | 1.3% |
| Totals | 1.5% | 0.5% | 1.7% |

analysis at 6 months (25° C.-60% RH):

| | | FORMULATION | |
|---|---|---|---|
| | | according to | CLASSIC |
| IMPURITIES | LIMITS | the invention | FORMULATION |
| Known | 0.5% | 0.4% | 0.6% |
| Unknown | 1% | 0.3% | 1.1% |
| Totals | 1.5% | 0.7% | 1.8% |

### Example 3

Core

The following gelled composition is prepared:

| COMPONENT | AMOUNT (g) |
|---|---|
| Polyvinylpyrrolidone | 90 |
| Polysorbate | 400 |
| Water | 510 |
| Total | 1000 |

dissolving Polyvinylpyrrolidone in water, to then add Polysorbate on dissolution. Mesalazine raw material is mixed with the above-mentioned-mentioned solution, to then extrude (600 μm net) and spheronize the compound. Immediately following spheronization, the granules obtained are dried in a Fluid Bed with inlet air at 80° C., up to a product temperature of 50° C. Cores having an average diameter of 570 μm-640 μm are obtained with the following dry matter composition:

| COMPONENT | AMOUNT (g) |
|---|---|
| Mesalazine | 985 |
| Polyvinylpyrrolidone | 2.75 |
| Polysorbate | 12.25 |
| Total | 1000 |

First Protective Coating

A protective membrane is prepared, comprising as follows:

| COMPONENT | AMOUNT (g) |
|---|---|
| Ethyl cellulose | 50 |
| Acetone | 950 |
| Total | 1000 |

By dissolving Cellulose in Acetone.

The protective membrane is sprayed onto the previously obtained cores using a Glatt Fluid Bed with Wurster insert, up to a weight increase of 0.8% with respect to the initial weight of the pellets, thus obtaining a compound with a Mesalazine content of 97.72%.

Second Gastro-Resistant Coating

A protective membrane is prepared, comprising as follows:

| COMPONENT | AMOUNT (g) |
|---|---|
| Eudragit FS 30 D | 180 |
| Talc | 18 |
| Triethyl citrate | 36 |
| Water | 766 |
| Total | 1000 |

Homogenising Triethyl citrate and Talc in water, then adding Eudragit to the compound.

The gastro resistant membrane is sprayed onto the previously obtained cores using a Glatt Fluid Bed with Wurster insert, up to a weight increase of 7% with respect to the initial weight of the pellets, thus obtaining a compound with a Mesalazine content of 91.33%.

The Finished Product has the following characteristics:

| | |
|---|---|
| Titre | 91.33% |

| | | |
|---|---|---|
| Average diameter | d(50) = 586 μm | d(90) = 624 μm |

| | |
|---|---|
| Density | 0.81 g/ml |

The pellets are analysed in-vitro in HCl 0.1N/750 ml for 2 h, and then adjusted to pH 7.2/1000 ml; the following dissolution profile is obtained:

| | PERCENTAGE | |
|---|---|---|
| | RELEASE of the | |
| | multi-layer pellets | |
| | according to | DISSOLUTION |
| TIMEFRAMES | the invention | SPECIFICATIONS |
| 2 h in HCl 0.1N | 1.4% | <10% |
| pH ADJUSTMENT | | |
| 30′ in pH 7.2 | 66.4% | N.L.T. 60% |
| 60′ in pH 7.2 | 95.2% | N.L.T. 85% |

Stability studies of the pellets according to the present invention are set up, in a climatic chamber with Temperature of 25° C. and Relative Humidity of 60%, to verify the development of impurities. After 3 to 6 months the following results are obtained and are compared with the stability results of a formulation obtained according to traditional composition and production methods:
analysis at 3 months (25° C.-60% RH):

| | | FORMULATION | |
|---|---|---|---|
| | | according to | CLASSIC |
| IMPURITIES | LIMITS | the invention | FORMULATION |
| Known | 0.5% | 0.2% | 0.4% |
| Unknown | 1% | 0.6% | 1.3% |
| Totals | 1.5% | 0.8% | 1.7% |

analysis at 6 months (25° C.-60% RH):

| | | FORMULATION | |
|---|---|---|---|
| | | according to | CLASSIC |
| IMPURITIES | LIMITS | the invention | FORMULATION |
| Known | 0.5% | 0.3% | 0.6% |
| Unknown | 1% | 0.6% | 1.1% |
| Totals | 1.5% | 0.9% | 1.8% |

1. A Granule containing Mesalazine as Active Pharmaceutical Ingredient (API), said granule consisting of API in mixture with a dried gelled composition in a ratio from 97:3 to 99:1, referred to the dry portion of the composition; said granule being obtained by extrusion, spheronization and drying of a mixture of the API with a gelled composition consisting of a mixture of 5-10% Polyvinyl pyrrolidone, 20-40% Polysorbate and 45-75% Water, where % refers to the percentages by weight with respect to the total weight of the gelled composition.

2. The granule according to claim 1 having an average size from 0.4 mm to 2 mm.

3. A pharmaceutical formulation comprising one or more granules according to claim 1.

4. A multi-layer pellet comprising the granule according to claim 1 as the inner core.

5. The multi-layer pellet according to claim 4, said pellet comprising:
an inner core consisting of the granule according to claim 1;
a first inner coating membrane, surrounding and contacting the core, said first membrane being pH-independent and comprising a polymer derived from cellulose, dissolved in non-aqueous solvent;
a second outer coating membrane, surrounding the first membrane, said second membrane being gastroprotective pH-dependent and comprising a polymer derivative of methacrylic acid selected from the anionic polymers with methacrylic acid as the functional group.

6. The pellet according to claim 5, wherein the first membrane comprises Ethyl cellulose having 3-110 cps viscosity, and the non-aqueous solvent is selected from Acetone, Ethanol and mixtures thereof; wherein the ethyl cellulose viscosity was measured on 5% solutions in Toluene/Ethanol (80%:20%) measured at 25° C. in a Ubbelohde viscometer.

7. The pellet according to claim 5, wherein the second membrane comprises a polymer selected from Eudragit L100-55, Eudragit L 30 D-55, Eudragit L100, Eudragit L12,5, Eudragit S100, Eudragit S12,5, Eudragit FS 30 D or mixtures thereof.

8. A pharmaceutical formulation for oral administration of Mesalazine, said formulation comprising the pellets according to claim 4.

9. A pharmaceutical formulation according to claim 8, said formulation being in the form of a sachet, tablet or capsule.

10. A process for preparing the granule according to claim 1, said process comprising preparing the gelled composition by dissolving Polyvinyl pyrrolidone in water first; and then, upon achieved dissolution, adding Polysorbate to achieve the gelification; the above-mentioned gelled composition is then added and mixed to Mesalazine, and finally the compound is extruded, spheronized and dried.

11. A process for preparing the multi-layer pellet according to claim 4, said process comprising:
coating the inner core with the first membrane in Fluidized Bed or in Coating Pan until achieving an increase in weight from 0.2% to 2% of dry portion with respect to the weight of the cores;
coating with the second membrane in Fluidized Bed or in Coating Pan until achieving an increase in weight from 5% to 15% of dry portion with respect to the weight of the cores.
